(19) United States
(12) Patent Application Publication
(10) Pub. No.: US 2006/0100589 A1
(43) Pub. Date: May 11, 2006

(54) Syringe safety sleeve

(75) Inventor: Woody Lin, Taipei (TW)

Correspondence Address: BACON & THOMAS, PLLC, 625 SLATERS LANE, FOURTH FLOOR, ALEXANDRIA, VA, 22314, US

(73) Assignee: Biotop Technology Co. Ltd., Taipei (TW)

(21) Appl. No.: 10/914,157

(22) Filed: Aug. 10, 2004

(51) Int. Cl. A61M 5/32

(52) U.S. Cl. 604/198

(57) ABSTRACT

A syringe safety sleeve has an inner sleeve and an outer sleeve. The inner sleeve selectively attaches to a conventional syringe and is slidably mounted inside the outer sleeve. The outer sleeve has an open proximal end and an open distal end. The inner sleeve has an open proximal end, an open distal end and a barrel connector formed on the open distal end. The open proximal end of the outer sleeve is selectively connected to the open proximal end of the inner sleeve when the outer sleeve is pulled out from the inner sleeve. The barrel connector of the inner sleeve attaches to a conventional syringe. The syringe safety sleeve can accommodate ordinary syringes a safety device to protect the used needle. The syringe safety sleeve can be conveniently connected to the syringe and has a simple structure.

### BACKGROUND OF THE INVENTION

1. Field of the Invention

The present invention relates to a safety feature for a syringe, and more particularly to a syringe safety sleeve that can be mounted around the barrel of a syringe and keep people from being injured by the needle of the syringe.

2. Description of Related Art

A conventional syringe comprises a hollow barrel, a plunger and a needle hub with a needle. To keep people from being injured by a used needle, manufacturers have developed safety syringes with retractable needles. In one configuration, the contaminated needle is drawn entirely into the barrel after the syringe has been used. In another configuration, a safety sleeve is formed with the syringe when the syringe is manufactured. The conventional safety sleeve comprises an outer sleeve and an inner sleeve slidably mounted inside the outer sleeve. The inner sleeve of the conventional safety sleeve is formed with the syringe, and the outer sleeve can be pulled out from the inner sleeve to retain the used needle.

However, conventional syringes with retractable needles or the safety sleeve formed with the syringe are directly modified during manufacturing and are expensive to manufacture. Furthermore, modified syringes cannot be used with ordinary syringes that have no safety device to protect the used needle.

Moreover, U.S. Pat. No. 6,344,032 discloses a disposable self-shielding unit dose syringe guard with a shield that is used for covering a needle thereof after medication is dispensed from the syringe. The conventional guard has a finger grip on a proximal end thereof which holds an ordinary syringe therein. The finger grip generally comprises a pair of wing-like members molded onto the proximal end of the guard. Each wing-like member includes a distal surface and an outer gripping surface extending proximally from an outer edge of the distal surface. Lateral surfaces extend proximally from the distal surfaces. The lateral surfaces of the finger grip include multiple detents partially defining a slot for holding the syringe inserted in the guard. A flange extended radially from the syringe must press the detents by force and hold in the slot. However, not all types of the flange of conventional syringes are suitable for the slot and the holding way of the syringe to the guard is inconvenient.

Another conventional syringe safety sleeve for attaching a conventional syringe comprises an inner sleeve and an outer sleeve. The inner sleeve is slidably mounted inside the outer sleeve and attaches to the barrel of a conventional syringe. The outer sleeve can pull out from the inner sleeve to retain the needle in the outer sleeve. However, the way of the syringe attaches to the barrel is inconvenient and not all types of the conventional syringes are suitable for the conventional syringe safety sleeve.

### SUMMARY OF THE INVENTION

The main objective of the present invention is to provide a syringe safety sleeve that is compatible with ordinary syringes with a simple structure and no safety device is needed to cover used needles of the syringes.

To achieve the objective, a syringe safety sleeve in accordance with the present invention comprises an inner sleeve and an outer sleeve. The inner sleeve is slidably mounted inside the outer sleeve and securely attaches to the barrel of a conventional syringe. The syringe safety sleeve can readily convert ordinary syringes lacking a needle safety device to a safety syringe. The conventional syringe can connect with the syringe safety sleeve easily. Furthermore, the syringe safety sleeve can suit conventional syringes with different sizes.

Further benefits and advantages of the present invention will become apparent after a careful reading of the detailed description with appropriate reference to the accompanying drawings.

### BRIEF DESCRIPTION OF THE DRAWINGS

FIG. 1 is an exploded perspective view of a first embodiment of a syringe safety sleeve in accordance with the present invention and a conventional syringe;

FIG. 2 is a side view in partial section of the syringe safety sleeve in FIG. 1 mounted on a conventional syringe;

FIG. 3 is another side view in partial section of the syringe safety sleeve and the conventional syringe in FIG. 2;

FIG. 4 is an operational top view of the syringe safety sleeve and the conventional syringe in FIG. 1 with a finger flange of the syringe rotated to be inserted into a barrel connector of an inner sleeve;

FIG. 5 is a top view of the syringe safety sleeve and the conventional syringe in FIG. 4 with the finger flange of the syringe inserted into the barrel connector;

FIG. 6 is a cross sectional view of the syringe safety sleeve and the conventional syringe in FIG. 3;

FIG. 7 is a side view of the syringe safety sleeve and the conventional syringe in FIG. 1 with an outer sleeve pulled to a top portion of the inner sleeve;

FIG. 8 is a second embodiment of a syringe safety sleeve in accordance with the present invention and a conventional syringe;

FIG. 9 is a side view in partial section of the syringe safety sleeve in FIG. 8 mounted on a conventional syringe; and

FIG. 10 is another side view in partial section of the syringe safety sleeve and the conventional syringe in FIG. 9.

### DETAILED DESCRIPTION OF THE INVENTION

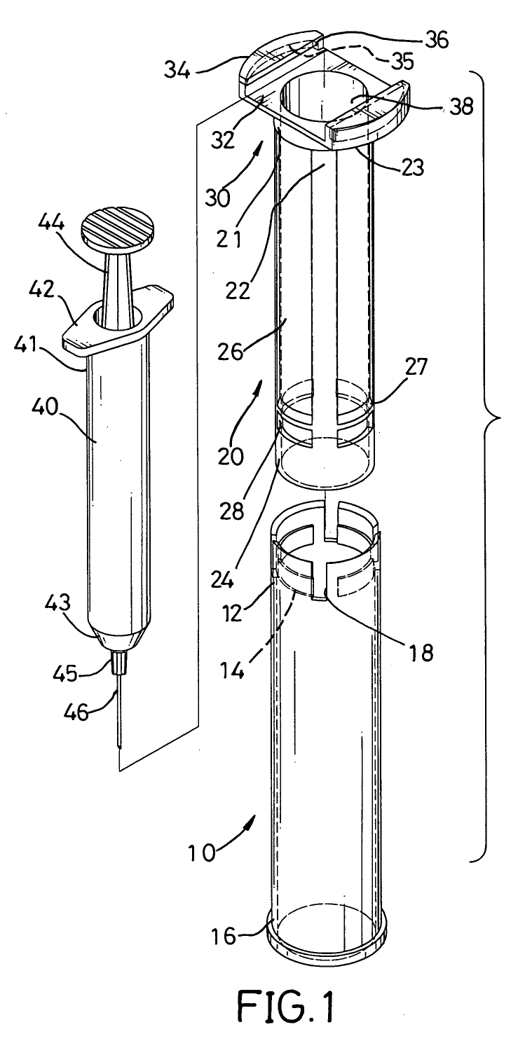

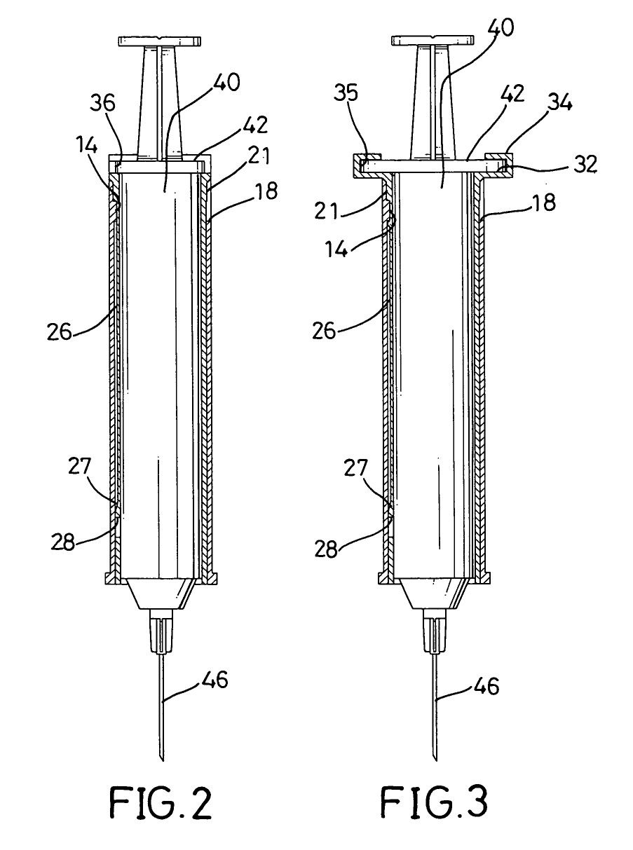

With reference to FIGS. 1 to 3 and 8, a syringe safety sleeve in accordance with the present invention is mounted on a conventional syringe (not numbered) to convert the conventional syringe to a safety syringe. The syringe comprises a hollow barrel (40), a plunger (44), a needle hub (45), a needle (46) and a safety cover (48). The hollow barrel (40) has a proximal end (41), a distal end (43) and a finger flange (42). The finger flange (42) is formed integrally with and extends radially out from the proximal end (41) of the hollow barrel (40). Before the syringe is used, the safety cover (48) covers the needle (46) and attaches to the needle hub (45).

The syringe safety sleeve in accordance with the present invention comprises an outer sleeve (10, 10′), an inner sleeve (20, 20′) and an optional cap (50).

In a first embodiment, the syringe safety sleeve comprises an outer sleeve (10) and an inner sleeve (20). The outer sleeve (10) has an open proximal end (12), an open distal end (16), multiple protrusions (14) and multiple optional slots (18). The multiple protrusions (14) extend inward from the open proximal end (12) and each has a bottom. The multiple optional slots (18) are formed in and extend longitudinally from the open proximal end (12) and intervene between the protrusions (14).

The inner sleeve (20) is slidably mounted inside the outer sleeve (10) and has an open proximal end (24), an open distal end (22), an optional protruded ring (21), multiple grooves (26), multiple side holes (28) and a barrel connector (30). The multiple side holes (28) are formed in and near the proximal end (24). The multiple grooves (26) are formed respectively corresponding to the side holes (28), formed in and extend longitudinally from the open distal end (22) and near the corresponding side hole (28) and each has a guiding inclined end (27) and a closed end (23). The guiding inclined end (27) is formed toward the corresponding side hole (28). The optional protruded ring (21) is formed around and extends radially from the open distal end (22). The open proximal end (24) is selectively connectable to the open proximal end (12) of the outer sleeve (10) by pulling the outer sleeve (10) out from the inner sleeve (20) until the multiple protrusions (14) engage the side holes (28).

The barrel connector (30) is formed on the open distal end (22) of the inner sleeve (20) to connect the inner sleeve (20) to a conventional syringe and has a base (32), two retainers (34) and a central hole (38). The base (32) is formed on and extends radially from the open distal end (22) and has a center and two ends. The two retainers (34) are formed respectively on and extend longitudinally from the end of the base (32) and each has two ends, an outer side, a chamber (35) and a positive limit (36). The outer side is arcuate. The chamber (35) is formed in the retainer (34). The positive limit (36) is formed on one end of the retainer (34). The two positive limits (36) of the two retainers (34) are formed at opposite directions. The central hole (38) is formed in the center of the base (32).

Before the syringe safety sleeve is mounted on a syringe, the open proximal end (24) of the inner sleeve (20) is compressed and inserted through the open proximal end (12) into the outer sleeve (10). The slots (18) of the outer sleeve (10) let the open proximal end (12) of the outer sleeve (10) expand to securely mount with the protruded ring (21) on the open distal end (22) of the inner sleeve (10).

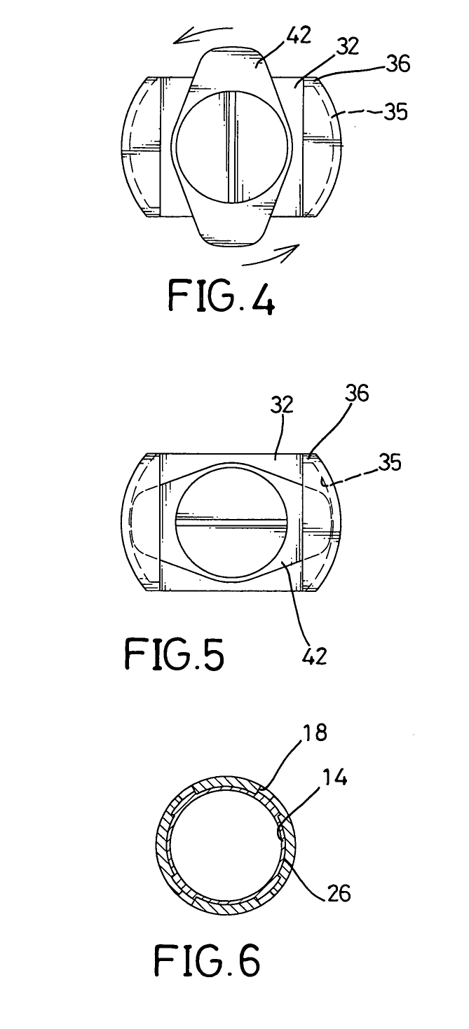

To mount the syringe safety sleeve on a syringe, the needle (46) with the safety cover (48) is inserted into the central hole (38) in the base (32) of the barrel connector (30). The barrel (40) of the syringe is pushed into the inner sleeve (20) until the finger flange (42) abuts the base (32) on the barrel connector (30). With further reference to FIGS. 4 and 5, where the finger flange (42) and the retainers (34) of the barrel connector (30) are staggered and then the finger flange (42) rotates to let the finger flange (42) inserted into the chambers (35) of the retainers (36) until the flange (42) is stopped by the positive limit (36). The barrel connector (30) is fastened around the finger flange (42) of the syringe and securely holds the syringe in the inner sleeve (20). When the finger flange (42) of the syringe is securely mounted in the connector (30) on the inner sleeve (20), the needle hub (45) of the syringe extends out of the open distal end (16) of the outer sleeve (10) when the open proximal end (12) of the outer sleeve (10) abuts the base (32) on the inner sleeve (20).

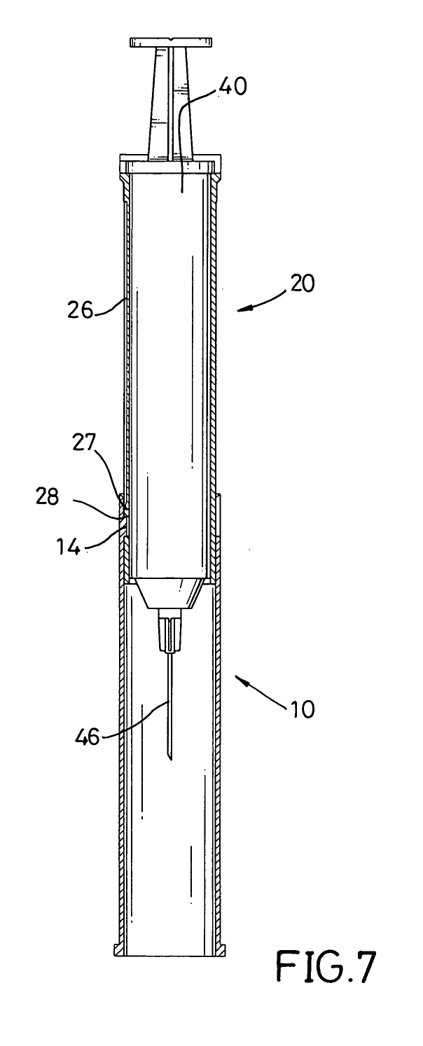

With further reference to FIGS. 6 and 7, to prevent personnel from being injured and infected by contaminated needles (46) during the disposal process, the outer sleeve (10) is pulled out from the inner sleeve (20) and locked in position to cover the contaminated needle (46). The open proximal end (12) of the outer sleeve (10) is locked to the open proximal end (24) of the inner sleeve (20) when the protrusions (14) on the outer sleeve (10) engage the side holes (28) on the inner sleeve (20). The protrusions (14) in the outer sleeve (10) move along the corresponding groove (26) and the guiding inclined end (27) of the groove (26) guide the protrusions (14) locked into the side holes (28).

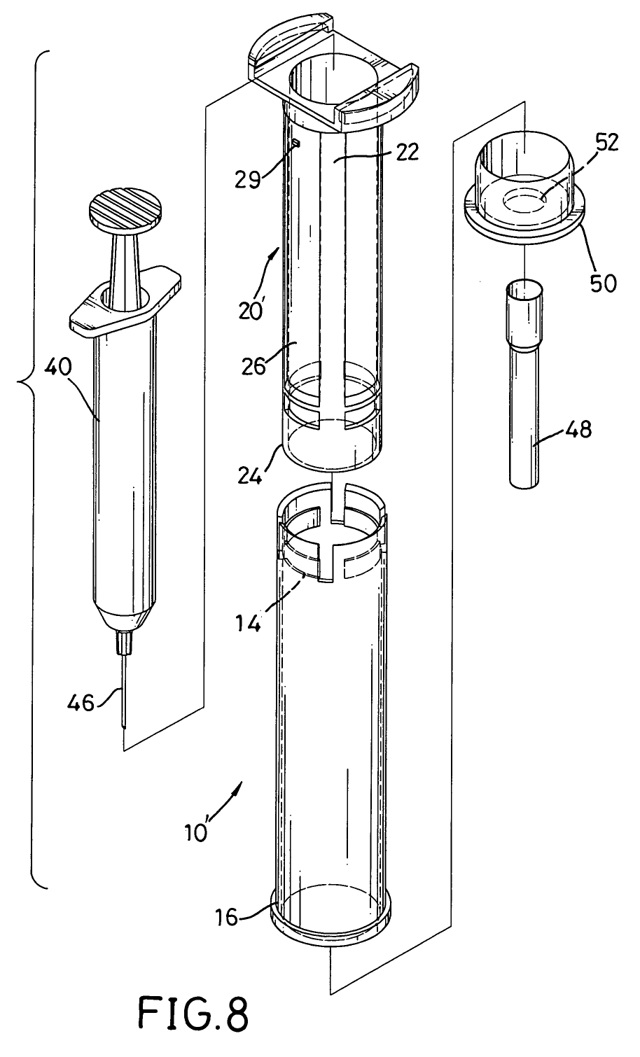

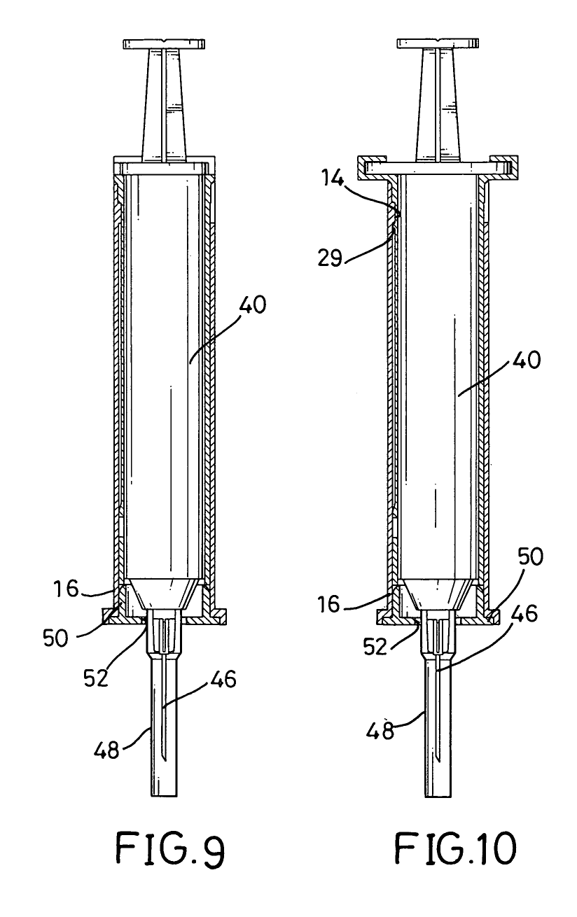

Referring to FIGS. 8, 9 and 10, a second embodiment of a syringe safety sleeve in accordance with the present invention comprises an outer sleeve (10′), an inner sleeve (20′) and a cap (50). The inner sleeve (20′) compared with the first embodiment further has multiple protruded limits (29). The multiple protruded limits (29) are formed on and extend longitudinally from the corresponding groove (26) and near the open distal end (22) and corresponding to the bottom of the protrusion (14). When the outer sleeve (10′) is mounted around the inner sleeve (20′), the protruded limit (29) will prevent the protrusions (14) of the outer sleeve (10′) from moving toward the open proximal end (24) of the inner sleeve (20′).

The cap (50) has a center and a hole (52) formed in the center. The cap (50) is engaged with the open distal end (16) of the outer sleeve (10′). When the outer sleeve (10′) is mounted around the inner sleeve (20′), the safety cover (48) with the needle (46) is protruded from the hole (52) of the cap (50). After the outer sleeve (20′) is pulled out from the inner sleeve (10′), the cap (50) will prevent the user's fingers from entering the outer sleeve (20′) and thus prevent accidental pricking.

The syringe safety sleeve for the syringe as described can accommodate an ordinary syringe priorly lacking a safety device to protect people from used needles. The syringe safety sleeve can be conveniently connected to the syringe and has a simple structure. Furthermore, the chamber of the retainer can suit different sizes of finger flange of a conventional syringe.

Although the invention has been explained in relation to its preferred embodiment, many other possible modifications and variations can be made without departing from the spirit and scope of the invention as hereinafter claimed.

What is claimed is:

1. A syringe safety sleeve mounted on a syringe comprising a hollow barrel having a proximal end, a distal end and a finger flange formed integrally with and extending radially out from the proximal end, a plunger, a needle hub, a needle and a safety cover covering the needle and attached to the needle hub before the syringe is used, the syringe safety sleeve comprising:
an outer sleeve having
an open proximal end;
an open distal end; and
multiple protrusions extended inward from the open proximal end and each having a bottom;
an inner sleeve slidably mounted inside the outer sleeve and having
an open proximal end selectively connected to the open proximal end of the outer sleeve by pulling the outer sleeve out from the inner sleeve;
an open distal end;
multiple side holes formed in and near the proximal end;
multiple grooves formed respectively corresponding to the side hole, formed in and extending longitudinally from the open distal end and near the corresponding side hole and each having
a guiding inclined end formed toward the corresponding side hole; and
a closed end; and
a barrel connector formed on the open distal end of the inner sleeve to connect the inner sleeve adapted to a syringe and having
a base formed on and extending radially from the open distal end and having a center and two ends;
two retainers formed respectively on and extending longitudinally from the end of the base and each having
two ends;
an outer side;
a chamber formed in the retainer; and
a positive limit formed on one end of the retainer, wherein the two positive limits of the two retainers are formed at opposite directions; and
a central hole formed in the center of the base.

2. The syringe safety sleeve as claimed in claim 1, wherein the outer sleeve further has multiple slots formed in and extending longitudinally from the open proximal end and intervening between the protrusions.

3. The syringe safety sleeve as claimed in claim 1, wherein inner sleeve further has a protruded ring formed around and extending radially from the open distal end of the inner sleeve.

4. The syringe safety sleeve as claimed in claim 1, wherein the outer side of the retainer on the barrel connector is arcuate.

5. The syringe safety sleeve as claimed in claim 1, wherein the inner sleeve further has multiple protruded limits formed on and extending longitudinally from the corresponding groove and near the open distal end and corresponding to the bottom of the protrusion.

6. The syringe safety sleeve as claimed in claim 1 further comprising a cap engaged with the open distal end of the outer sleeve and having a center and a hole formed in the center.
